(19) United States
(12) Patent Application Publication
(10) Pub. No.: US 2014/0066755 A1
(43) Pub. Date: Mar. 6, 2014

(54) Simultaneous Imaging and Particle Therapy Treatment system and Method

(71) Applicants: Joe Matteo, Walland, TN (US); Tony Brun, Knoxville, TN (US); Terry Douglass, Knoxville, TN (US)

(72) Inventors: Joe Matteo, Walland, TN (US); Tony Brun, Knoxville, TN (US); Terry Douglass, Knoxville, TN (US)

(73) Assignee: ProNova Solutions, LLC, Knoxville, TN (US)

(21) Appl. No.: 13/597,875

(22) Filed: Aug. 29, 2012

(51) Int. Cl. A61B 6/03; A61N 5/10; A61B 6/00

(52) U.S. Cl. 600/427

(57) ABSTRACT

A simultaneous imaging and particle therapy treatment system including a means for generating a particle beamline, a treatment bed to receive and support a patient having a treatment volume, a gantry to receive the particle beamline from the generating means and to redirect the beamline to the patient's treatment volume, the gantry rotating about the treatment bed with an axis of rotation substantially coplanar with the treatment bed and redirecting the beamline to encounter the treatment volume substantially perpendicular to the gantry's axis of rotation, and an image scanner having a plurality of detector arrays radially positioned around the treatment bed to capture images of the treatment volume; whereby the scanner and gantry simultaneously capture images of and treat the treatment volume with particle therapy.

### CROSS-REFERENCE TO RELATED APPLICATIONS

N/A

### FIELD OF INVENTION

The present inventive concept relates generally to cancer treatment technology, and more particularly to a system and method to simultaneously capture images, such as computed tomography and/or positron emission tomography images, and treat a patient with particle therapy.

### BACKGROUND

The use of particle therapy in cancer treatment has been known in the art. Particle therapy generally includes a series of energized particles, such as protons, directed to a tumor, or treatment volume, in a patient's body. Particles may be generated in a particle accelerator, commonly referred to as a cyclotron and/or a synchrotron, and directed to the patient in the form of a beamline using a series of magnets that guide and shape the particle beamline such that the particles penetrate the patient's body at a selected location and are deposited at the site of the treatment volume. Particle therapy leverages the Bragg Peak property of charged particles such that the majority of the energy is deposited within the last few millimeters of travel along the beamline—at a point commonly referred to as the isocenter, as opposed to conventional, intensity modulated radiation therapy (i.e., photons) in which the majority of energy is deposited in the first few millimeters of travel, thereby undesirably damaging healthy tissue.

Particle therapy treatment facilities typically consist of a single cyclotron and a plurality of treatment rooms. Thus, the single cyclotron is often adapted to generate a particle beamline that is then selectively directed to one of the various treatment rooms. A particle therapy treatment may include the selection of a desired energy level for the beamline, such that the energy of the particles is deposited substantially at the desired location (i.e., the treatment volume) inside the patient's body. Therefore, the energy level selection is directly related to the position and shape of the treatment volume within the patient's body. Frequently, the cyclotron will generate a standard high-energy beamline, which may then be selectively modified as desired for the particular treatment protocol.

The beamline may be directed immediately to the patient without the need for any redirection. However, a more common approach is to redirect the beamline using a series of cooperating bending magnets commonly referred to as a gantry. FIG. 1A illustrates an example embodiment prior art particle therapy gantry designed to receive and redirect a particle beamline to a patient. As illustrated, the particle therapy gantry 21 includes at least three bending magnets 11A-C to redirect the particle beamline 15 to the gantry's treatment nozzle 13, and eventually the patient 9 positioned on a treatment bed 17. This allows the beamline 15 to be selectively directed to the patient 9 from any angle and permits a physician to design a treatment plan that minimizes undesirable effects on healthy tissue. Stated differently, gantries are frequently adapted to rotate about a patient, and redirect the beamline to be perpendicular to the gantry's axis of rotation 19, illustrated by the directional arrow 19′ in FIG. 1. Thus, the treatment nozzle 13 and beamline 15 may be rotated about the patient 9 such that the beamline 15 is able to penetrate the patient's body at a plurality of locations and encounter the treatment volume from multiple directions. This minimizes adverse effects on healthy tissue and increases the efficacy of the treatment.

Typically, a patient may undergo particle therapy by receiving a series of daily treatments over the course of several weeks. Each treatment, however, requires anatomical imaging prior to initiation of the beamline to confirm and/or verify the position of the treatment volume. This imaging is frequently done using basic, planar computed tomography (CT) x-ray technology.

Particle irradiation also causes certain nuclear reactions involving stable isotopes in the body. For example, particle therapy exposure frequently generates positron emitting isotopes in the patient's body, including Oxygen-15, Carbon-11, Nitrogen-13, and/or Flourine-18. These positron emitting isotopes can then be imaged using a Positron Emission Tomography (PET) scanner following a particle therapy treatment. Physicians may use these images, frequently in combination with post-treatment CT images, to verify the accuracy of the previous treatment.

FIG. 1B illustrates a cross-sectional view of an example embodiment prior art CT/PET image scanner. As illustrated, the image scanner 41 includes a toroidal frame, through which a treatment bed 17′ supporting a patient 9′ may be received. The image scanner may contain certain detector arrays radially positioned around the treatment bed 17′ to capture CT and/or PET images. The example embodiment illustrated in FIG. 1 includes an x-ray tube 53 to dispense x-ray beams 57A-E at the patient 9′ having a treatment volume 25. After encountering the patient 9, the x-ray beams are received by an x-ray detector array 55. Likewise, PET detector arrays 59A and 59B are adapted to receive the positrons 61 emitted from isotopes in the body that have undergone nuclear reactions due to a prior particle therapy treatment. These imaging components may be adapted to rotate about the patient, within the toroidal frame of the image scanner 41, in order to capture effective images. Thus, with a beamline being redirected to encounter the patient, known imaging devices are not adapted to cooperate with a particle therapy gantry to capture images simultaneously with particle therapy treatment.

In light of the above, there exists a need in the art for systems and methods to simultaneously capture images of and treat a patient with particle therapy. Simultaneous imaging and treatment will increase the efficacy of the treatment, as well as decrease the adverse effects on healthy tissue by allowing physicians to more accurately target the treatment volume in the patient's body.

### BRIEF SUMMARY

The present general inventive concept provides a system and method whereby a patient containing a tumor may simultaneously receive particle therapy treatment and imaging, such as CT imaging, PET imaging, or both.

In accordance with various embodiments of the present general inventive concept, a simultaneous imaging and particle therapy treatment system may include a means for generating a particle beamline, a treatment bed to receive and support a patient having a treatment volume, a gantry receiving the particle beamline from the generating means and redirecting the beamline to the patient's treatment volume, the gantry rotating about the treatment bed with a rotational axis substantially coplanar with the treatment bed and redirecting the beamline to encounter the treatment volume substantially perpendicular to the gantry's axis of rotation, an image scanner having a plurality of detector arrays radially positioned around the treatment bed to capture images of the treatment volume; and whereby the scanner and gantry simultaneously capture images of and treat the treatment volume with particle therapy.

In some embodiments, the image scanner captures CT images, PET images, or both.

In some embodiments, the system further includes a positioning means provided to the scanner to selectively position the scanner relative the treatment bed. In some embodiments the sliding means includes a track provided substantially parallel to the treatment bed, the scanner coupled to the track, and a transport device coupled to the scanner to selectively position the scanner along the track. In other embodiments, the positioning means includes a non-linear actuator.

In some embodiments, the image scanner captures images of the treatment volume while the beamline is encountering the treatment volume.

In some embodiments, the image scanner includes at least one treatment port through which the beamline passes to encounter the treatment volume.

In some embodiments, the system further includes an environmental chamber interposing the gantry and the patient through which the beamline passes before encountering the treatment volume. In some embodiments, the system further includes a helium source in fluid communication with the environmental chamber. In some embodiments, the system further includes a vacuum device in fluid communication with the environmental chamber. In some embodiments, the environmental chamber is coupled to the gantry and is selectively positionable relative the gantry between a first extended position and a second retracted position.

In some embodiments, the centerpoint of the plurality of radially positioned detector arrays is the treatment volume and at least one detector array is positionable adjacent the redirected beamline while the beamline is encountering the treatment volume.

In some embodiments, the gantry further defines an isocenter, the isocenter being the intersection of the redirected particle beamline and the gantry's axis of rotation, the scanner being positionable such that the isocenter is the centerpoint of the plurality of radially positioned detector arrays.

In some embodiments, the image scanner occupies a position such that the isocenter is the centerpoint of the plurality of radially positioned detector arrays while the particle beamline is encountering the treatment volume.

In accordance with various example embodiments of the present general inventive concept, a simultaneous particle therapy treatment and imaging method includes positioning on a treatment bed a patient containing a treatment volume, the treatment bed cooperating with a gantry, the gantry receiving a beamline from a particle generator means and redirecting the beamline to the treatment volume, the treatment bed further cooperating with an image scanner having a plurality of detector arrays radially positioned around the treatment bed to capture images of the treatment volume while the patient is on the treatment bed; capturing a first image of the treatment volume using the image scanner; directing a particle beamline to encounter the treatment volume; capturing a second image of the treatment volume using the image scanner; and whereby the capturing a first image operation, the directing operation, and the capturing a second image operation are performed simultaneously.

In some embodiments, the gantry rotates about the treatment bed with an axis of rotation substantially coplanar with the treatment bed, the gantry further redirecting the particle beamline to encounter the treatment volume substantially perpendicular to the axis of rotation, the gantry further defining an isocenter, the isocenter being the intersection of the redirected particle beamline and the axis of rotation, the method further including positioning the scanner to occupy a first position relative the treatment bed such that the isocenter is the centerpoint of the plurality of radially positioned detector arrays; positioning the scanner to occupy a second position relative the treatment bed such that the isocenter is not the centerpoint of the plurality of radially positioned detector arrays; and whereby the taking a first image operation and the taking a second image operation are performed by the scanner while occupying the first position, and the directing operation is performed by the gantry while the scanner occupies the second position.

In some embodiments, the image scanner captures CT images, PET images, or both. In some embodiments, the first image is a CT image and the second image is a PET image.

In some embodiments, the capturing a second image operation is performed during the directing operation.

Additional aspects and advantages of the present general inventive concept will be set forth in part in the description which follows, and, in part, will be obvious from the description, or may be learned by practice of the present general inventive concept.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1A illustrates an example embodiment prior art particle therapy gantry designed to receive and redirect a particle beamline to a patient;

FIG. 1B illustrates a cross-sectional view of an example embodiment prior art CT/PET image scanner;

FIG. 2 illustrates a top down view of an example embodiment particle therapy facility including two treatment rooms each having a simultaneous imaging and particle therapy treatment system;

FIGS. 3 A&B illustrate side view representative diagrams of the example embodiment first treatment room included in FIG. 1, depicting a patient containing a treatment volume and positioned on the treatment bed to receive simultaneous imaging and particle therapy;

FIGS. 4 A&B illustrate perspective views of example embodiment simultaneous imaging and particle therapy systems whereby treatment ports have been provided to the image scanner to permit the particle beamline to penetrate the image scanner and encounter the treatment volume;

FIG. 4C illustrates a representative diagram of another example embodiment image scanner in accordance with the present general inventive concept;

FIG. 4D illustrates another example embodiment image scanner whereby the various imaging components are radially positioned around a treatment bed without being housed in a toroidal frame, in accordance with the present general inventive concept; and

FIG. 5 illustrates yet another example embodiment image scanner that has been provided with a sliding means to selectively position the image scanner relative the treatment bed, in accordance with the present general inventive concept.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures. The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art.

In accordance with various example embodiments of the present general inventive concept, a patient may simultaneously receive particle therapy treatment and imaging, such as CT imaging, PET imaging, or both. One of skill in the art will recognize that particle therapy may include, but is not limited to, proton therapy, carbon ion therapy, or other types of particle therapy whereby an energized particle beamline is generated and directed to a treatment volume. It will also be noted hereafter in this application that the term ‘simultaneously’ refers to two or more operations occurring either absolutely simultaneously (i.e., at the same time, also referred to herein using the terms ‘during’ and/or ‘while’) as well as substantially simultaneously (i.e., sequentially and/or consecutively while the patient remains on the same treatment bed).

While the example embodiments discussed and illustrated herein generally include CT and PET imaging, the present general inventive concept is not limited to CT and/or PET imaging. Furthermore, while example embodiments discussed and illustrated herein include spiral CT image scanners, one skilled in the art will recognize that the present general inventive concept is not limited to spiral CT image scanners. Other types of CT image scanners, including but not limited to cone beam scanning and electron beam tomography scanning, may be incorporated without departing from the scope or spirit of the present general inventive concept. Additionally, while the example embodiments discussed and illustrated herein are generally directed to Pencil Beam Scanning particle therapy (i.e., where a narrow beam of particles is dynamically targeted across the treatment volume, painting it layer by layer), it will be understood by those of skill in the art that the present general inventive concept is not limited to Pencil Beam Scanning particle therapy. Other types of particle therapy, including but not limited to scattering, may be incorporated, depending on the inclusion, type, and positioning of a treatment nozzle, without departing from the scope or spirit of the present general inventive concept.

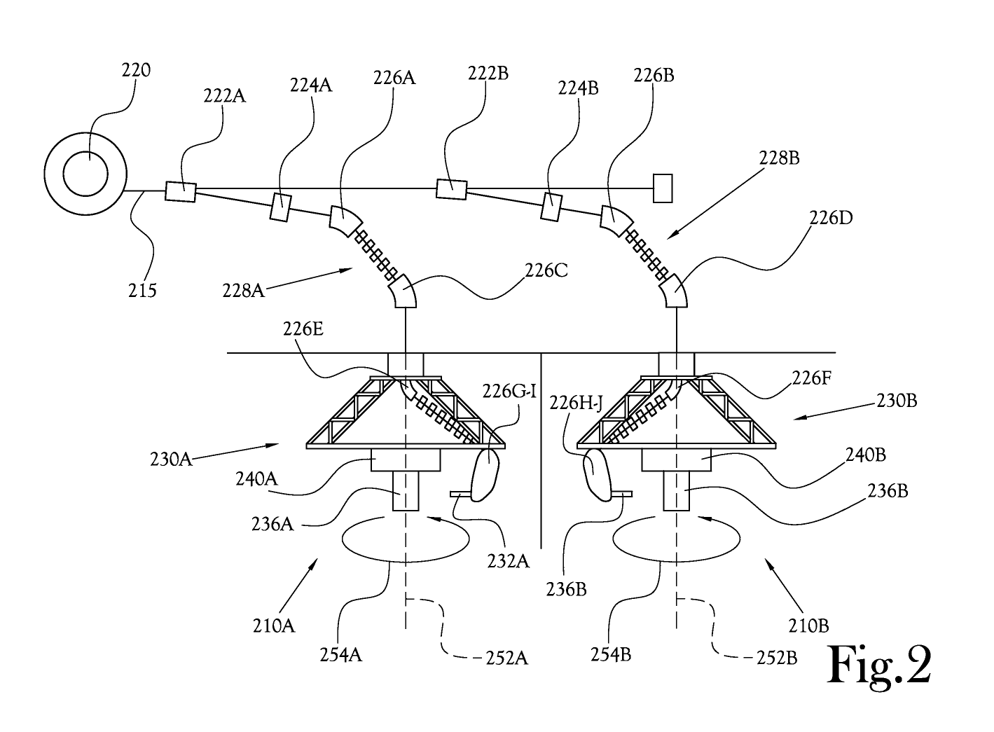

FIG. 2 illustrates a top down view of an example embodiment particle therapy facility. The illustrated example embodiment includes two treatment rooms 210A and 210B positioned adjacent one another. Provided to each treatment room is a simultaneous imaging and particle therapy treatment system, which may include a treatment bed 236A and 236B, an image scanner 240A and 240B, as well as a rotating gantry 230A and 230B. Each gantry's rotational orientation is illustrated by axes of rotation 252A and 252B, as well as the rotation directional arrows 254A and 254B. Each treatment room is adapted to accommodate a single patient having a treatment volume, such as a cancerous tumor.

A simultaneous imaging and particle therapy treatment system may also include a particle beamline generating means, such as a cyclotron or particle accelerator, as depicted at 220 in the illustrated example embodiment. The accelerator 220 produces a particle beamline 215 that is selectively directed to one of the treatment rooms 210A and/or 210B. Kicker magnets 222A and 222B are included to selectively modify the directional path of the particle beamline 215 from a straight line of travel, to an angular line of travel (thirty degrees in the illustrated example embodiment), thereby beginning the selective redirection of the beamline 215 to one of the two treatment rooms 210A and/or 210B. Stated differently, a particle beamline 215 is projected along a straight path substantially parallel to two consecutively adjacent treatment rooms 210A and 210B. The kicker magnets 222A and 222B are provided along the straight path at selected locations relative each treatment room 210A and/or 210B to selectively offset the directional path traveled by the beamline 215. Thus, the projected beamline 215 is either redirected by the first kicker magnet 222A, or permitted to continue along the straight path to the second kicker magnet 222B, where it will then be redirected.

Degraders 224A and 224B may be provided to degrade the particle beamline 215 to the desired energy level for the particular particle therapy treatment protocol. An energy selection system, depicted at 228A and 228B in the illustrated example embodiment, is also provided upstream of each treatment room 210A and 210B to filter out various particle energies that are output by the degrader, so as to only pass along a narrow range of energies for the treatment.

The particle beamline 215 may be redirected to the treatment rooms, and eventually the treatment volume, by bending magnets 226A-F. As illustrated in FIG. 2, bending magnets 226 A, C, & E cooperate to bend the beamline 215 in a desired direction toward the first treatment room 210A. More specifically, the first bending magnet 226A may bend the beamline 215 thirty degrees and the second bending magnet 226C may bend the beamline 215 another thirty degrees. Thus, when combining these directional changes with that achieved by the first kicker magnet 222A (thirty degrees), the particle beamline 215 may be redirected ninety degrees after passing through the second bending magnet 226, such that the particle beamline 215 is directed from the accelerator to the first treatment room 210A. One of skill in the art will recognize that numerous angles of redirection may be incorporated without departing from the scope or spirit of the present general inventive concept. Accordingly, the example angles of redirection disclosed herein are non-limiting.

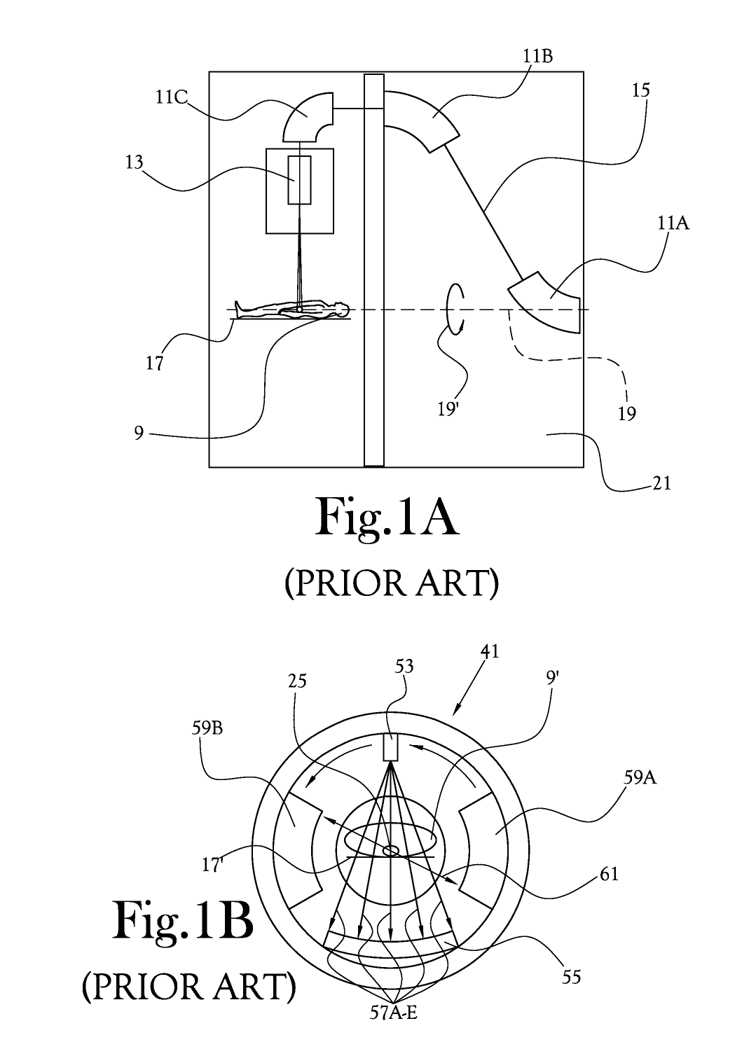

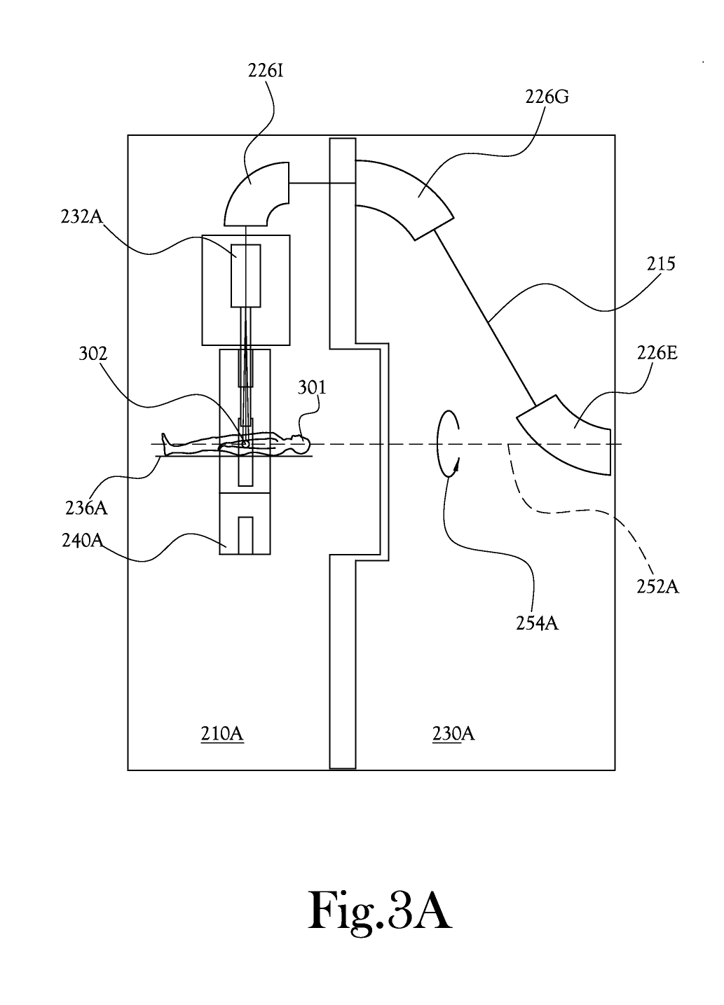

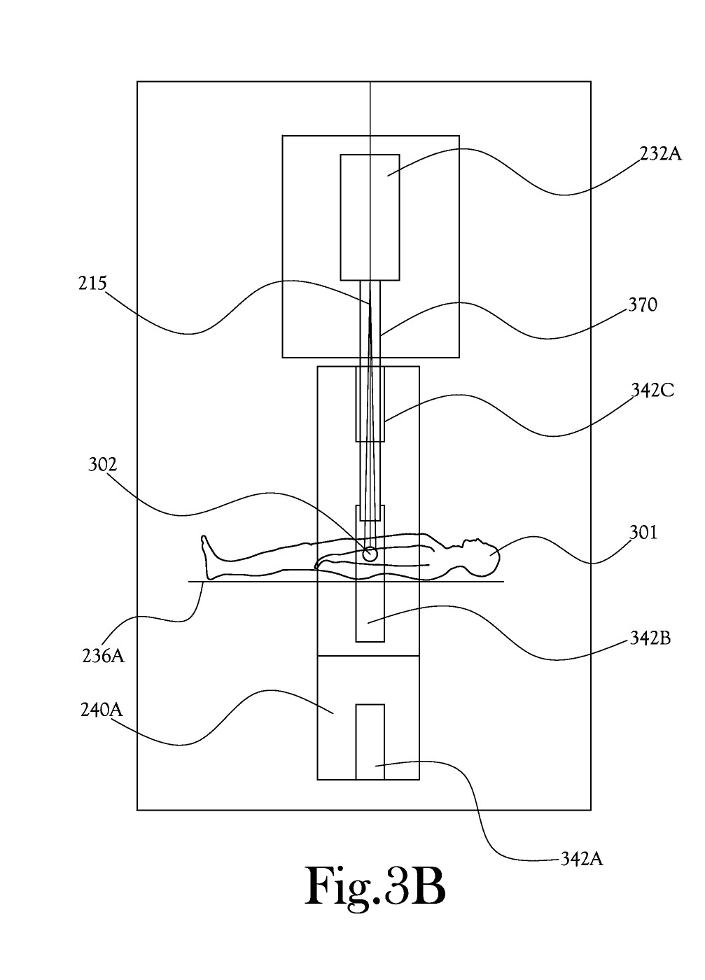

FIGS. 3 A&B illustrate side view representative diagrams of the example embodiment first treatment room 210A included in FIG. 1, depicting a patient containing a treatment volume and positioned on the treatment bed.

The particle beamline 215 has been redirected from the accelerator 220 (in FIG. 2) to the gantry 230A, where it encounters the first of the gantry's three bending magnets 226 E, G, & I. The gantry's first bending magnet 226E may redirect the beamline 215 vertically upwards by sixty degrees. The gantry's second bending magnet 226G may then redirect the beamline 215 sixty degrees vertically downwards, such that the beamline 215 is once again parallel to the ground. The gantry's third bending magnet 2261 may then redirect the beamline ninety degrees vertically downward, such that the beamline is now perpendicular to the ground and the treatment bed 236A. One of skill in the art will recognize that numerous angles of redirection may be incorporated without departing from the scope or spirit of the present general inventive concept. Accordingly, the example angles of redirection disclosed herein are non-limiting.

As indicated by the directional arrow 254A, the gantry may be adapted to rotate about an axis 252A to permit the beamline 215 to encounter a patient 301 from any angle within the rotational plane. It will be understood that in some embodiments the gantry will be able to rotate a full three hundred sixty degrees, whereas in other embodiments the gantry's range of rotation will be limited by certain factors, such as the floor and/or supporting means for the treatment bed. Hence, for reference in the present application, use of the term ‘rotate’ refers to a curved movement about a centerpoint, and not necessarily to a full circular movement (unless specified). Thus, the gantry may be adapted such that, regardless of the beamline's positioning within the rotational plane, the redirected beamline always encounters the patient 301 at an angle substantially perpendicular to the axis of rotation 252A. The redirected, perpendicular beamline 215 and axis of rotation 252A may intersect at an isocenter. In the illustrated embodiment, the patient's treatment volume 302 occupies the isocenter of the present example embodiment system.

A treatment nozzle 232A is provided just below the gantry's third bending magnet 2261. One of skill in the art will recognize that the precise location of the treatment nozzle may vary without departing from the scope or spirit of the present general inventive concept.

The image scanner 240A has been positioned in FIGS. 3 A&B proximate the treatment bed 236A. Treatment ports 342A-C have been provided in the image scanner 240A to permit the beamline 215 to pass therethrough. In the illustrated example embodiment, an environmental chamber 370 (see FIG. 3B) has also been provided to the gantry to reduce particle scatter as the beamline 215 travels from the treatment nozzle 232A to the treatment volume 302.

The environmental chamber 370, in the currently illustrated example embodiment, is an elongated enclosure designed to extend from a location proximate the treatment nozzle 232A to a location proximate the patient 301 and/or image scanner 240A. The environmental chamber 370 is adapted to reduce particle scatter as the beamline 215 travels from the treatment nozzle 232A to the treatment volume 232A. In some embodiments, the environmental chamber 370 is in fluid communication with a vacuum device to selectively initiate at least a partial vacuum within the environmental chamber 370 during particle therapy treatment. The vacuum device may be positioned proximate the treatment nozzle and selectively placed in fluid communication with the chamber 370 using a valve or other similar control means known in the art. In some embodiments, the environmental chamber 370 is in fluid communication with a helium supply means, which may include a helium source, such as a container, equipped with a control means, such as a valve, to selectively initiate a low pressure, helium-rich environment within the chamber 370.

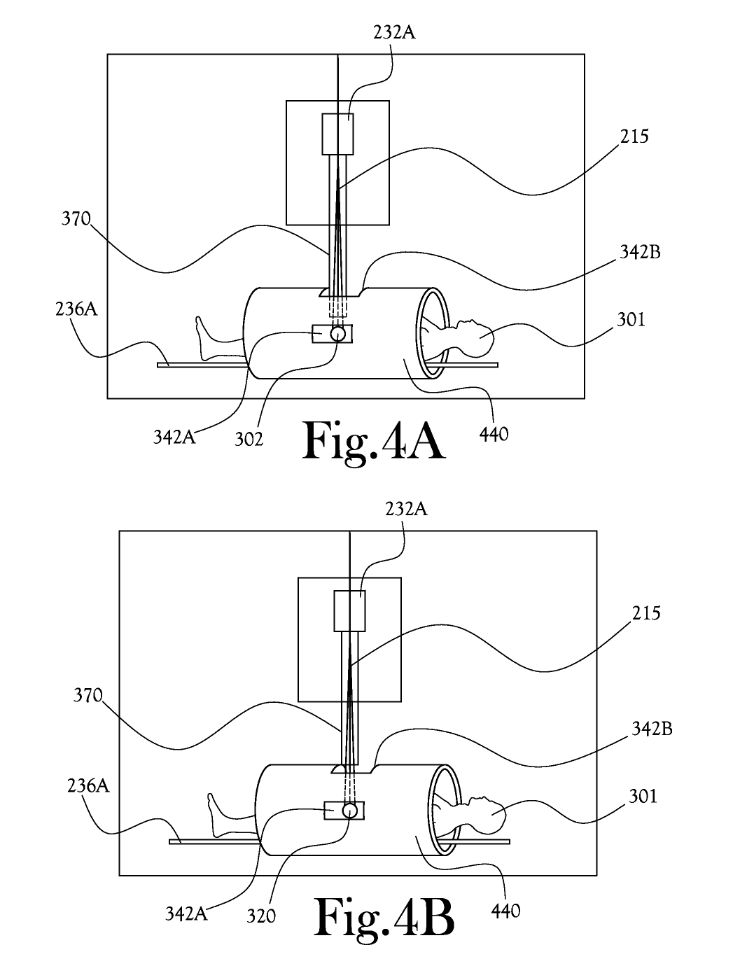

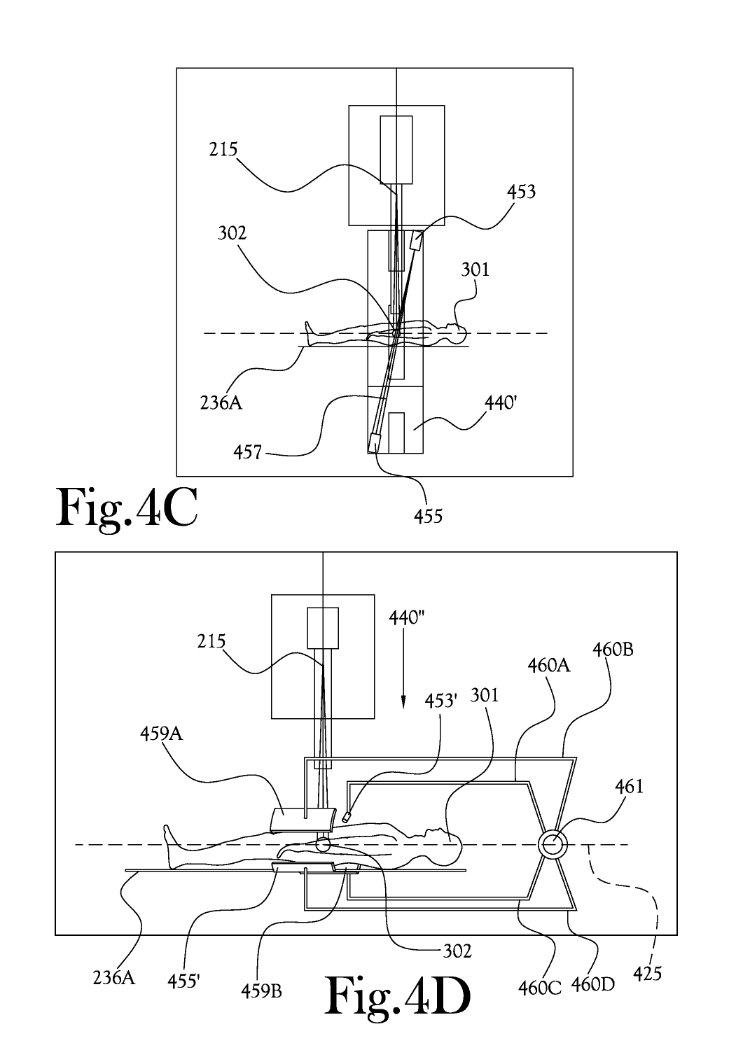

FIGS. 4 A&B illustrate perspective views of example embodiment simultaneous imaging and particle therapy systems whereby treatment ports have been provided to the image scanner to permit the particle beamline to penetrate the image scanner and encounter the treatment volume. In FIG. 4A, the environmental chamber has been extended from a position proximate the treatment nozzle 232A to a position proximate the treatment volume 302, thereby penetrating treatment port 342B of the image scanner 440.

Still referring to FIG. 4A, the system accommodates simultaneous imaging and particle therapy treatment. In some embodiments, a simultaneous imaging and particle therapy method may include the image scanner 440 capturing a first image of the treatment volume 302 without the environmental chamber 370 extended or the beamline 215 being directed to the treatment volume 302. This may be achieved by rotating the appropriate components within the image scanner 440, such as the x-ray tube and x-ray detector array, or by rotating the entire image scanner about the treatment bed 236A. Following the operation of capturing at least a first image, the environmental chamber 370 may be extended to penetrate the image scanner 440 through one of its treatment ports 342A-C. While the chamber 370 is deployed, the particle beamline 215 may be directed to the treatment volume 302 for particle therapy treatment. In some embodiments, the image scanner 440 is adapted to cooperatively rotate with the gantry/beamline 215 to accommodate particle therapy treatment of the treatment volume 302 from multiple directions. After treating the treatment volume 302 with particle therapy, the chamber 370 may be retracted such that it no longer penetrates the treatment port 342B, and the image scanner 440 may then perform the operation of capturing at least a second image, such as a PET image.

Still referring to FIG. 4A, in some embodiments the PET detector arrays are adapted to rotate in a limited manner within the image scanner such that each PET detector array only moves within a limited range of positions on opposing sides of the treatment volume 302, frequently referred to as ‘wobble rotation’. Thus, in some embodiments, the PET imaging and the particle therapy treatment may be absolutely simultaneous, even with the environmental chamber 370 penetrating the image scanner 440.

In FIG. 4B, the environmental chamber 370 has been extended from a position proximate the treatment nozzle 232A to a position proximate the treatment port 342B, but without penetrating the treatment port 342B. Thus, in some embodiments, the particle beamline 215 is adapted to treat the treatment volume 302 without the environmental chamber extending all the way to the patient 301. The current example embodiment system accommodates simultaneous imaging and particle therapy treatment such that the image scanner 440 may be rotated about the patient and the particle beamline 215 may be selectively and/or intermittently projected, or pulsed, through the treatment ports 342A-C.

FIG. 4C illustrates a representative diagram of another image scanner 440′ in accordance with an example embodiment of the present general inventive concept. In the illustration, the image scanner 440′ has a substantially toroidal frame through which the treatment bed 236A may be received. Inside the image scanner 440′, an x-ray tube 453 and detector array 455 are positioned diagonally with respect to the axis of rotation, yet directed toward the isocenter/treatment volume 302, such that the x-ray tube 453 and detector array 455 may rotate 360 degrees while the beamline 215 (and optional environmental chamber) is penetrating the image scanner 440′. Stated differently, the x-ray components are disposed within the image scanner 440′ in a non-coplanar fashion with respect to the beamline 215, thereby allowing the x-ray components to complete rotations while the beamline 215 enjoys an unobstructed path to the treatment volume 302. It will be noted that while the presently illustrated example embodiment includes an image scanner having only x-ray imaging components, that PET detector arrays may be similarly positioned within the image scanner without departing from the scope or spirit of the present invention.

FIG. 4D illustrates yet another example embodiment image scanner 440″ in accordance with example embodiments of the present general inventive concept. Instead of being contained within a toroidal frame, the imaging components are each radially positioned around the treatment bed 236A using arm members 460A-D. An x-ray tube 453′, x-ray detector array 455′, and two PET detector arrays 459 A&B may be radially positioned around the patient 301 and oriented such that the treatment volume 302 occupies the isocenter of the imaging components when in the illustrated positions. Arm members 460A-D, of the type known to those of skill in the art, are coupled to each imaging component and to a position control means, such as a centralized actuator 461, also of the type known to those of skill in the art. Thus, the imaging components may be selectively positioned proximate the treatment volume 302, and rotated about the rotational axis 425 using the arm members 460A-D and position control means 461. In the illustrated embodiment, it will be noted that the rotational axis 425 and treatment bed 236A are substantially coplanar.

Still referring to FIG. 4D, each of the operations for performing simultaneous imaging and particle therapy treatment may be practiced by the currently illustrated example embodiment. Briefly, the gaps between each image capturing component are adapted to function in a similar way as the treatment ports in the previous example embodiments. Additionally, however, the presently illustrated example embodiment image scanner 440″ may also be adapted to selectively position the imaging components in a first position proximate the treatment volume, as illustrated, to capture at least a first image of the treatment volume, such as a CT image. Following the operation of capturing the first image, the components may then be selectively repositioned to occupy a second position distal the treatment volume to allow for particle therapy treatment. After particle therapy treatment, the components may again be positioned to occupy the first position proximate the treatment volume to take at least a second image, such as a PET image.

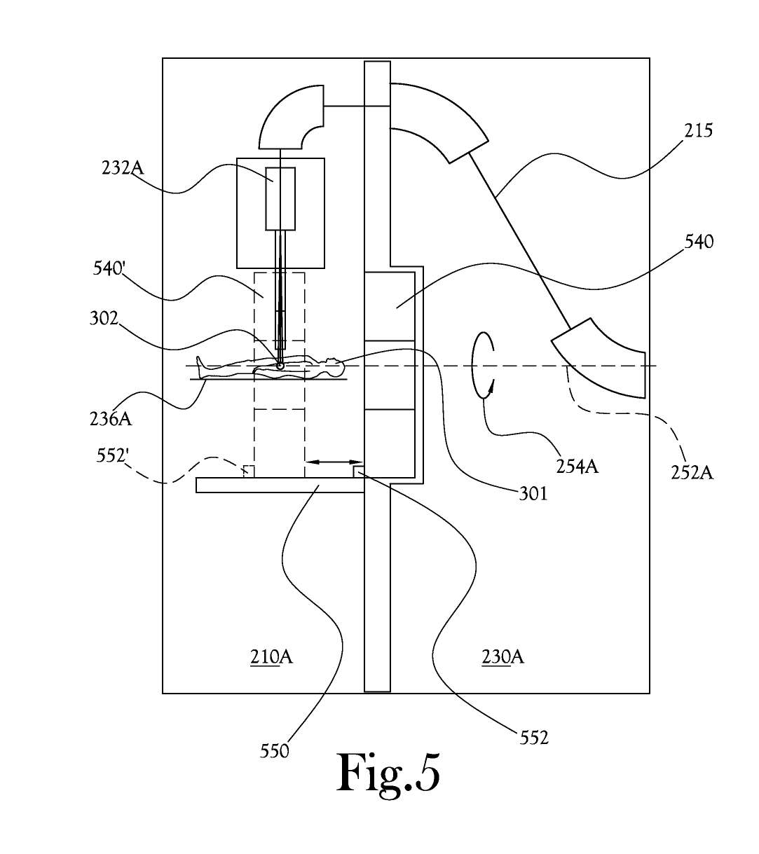

FIG. 5 illustrates another example embodiment image scanner that has been provided with a sliding means to selectively position the image scanner relative the treatment bed. The treatment room 210A includes a gantry 230A and an image scanner 540 coupled to a track 550. The track 550 is substantially parallel and below the treatment bed 236A. One skilled in the art will recognize that the specific location of the track 550 may be modified without departing from the scope or spirit of the present general inventive concept. Coupled to the image scanner 540 is a transport device, depicted at 552 and 552′. The transport device 552 is adapted to selectively position the image scanner 540 along the track 550. Thus, as illustrated using dotted lines, the image scanner 540′ is occupying a first position such that it is interposing the treatment nozzle 232A and the patient 301 to orient its detector arrays such that the treatment volume occupies the isocenter of the imaging components. Hence, while the image scanner 540′ is occupying the first position, the particle beamline 215 is either penetrating the image scanner 540′, such as through a treatment port (not illustrated), or is not being directed to the treatment volume 302.

It will be noted that one of skill in the art will recognize that numerous other means may be provided for selectively positioning the image scanner 540 relative the treatment bed 236A and/or beamline 215. For instance, a robot or other non-linear/multi-degree-of-freedom actuator may be provided and operatively coupled to the image scanner 540 to selectively position the image scanner 540.

Further, as illustrated using solid lines, the image scanner 540 may occupy a second position along the track 550 such that it is not interposing the gantry's treatment nozzle 232A and the patient 301. Stated differently, when the image scanner 540 is occupying the second position, the particle beamline 215 may encounter the treatment volume 302 without having to penetrate the image scanner 540. One of skill in the art will recognize that the present general inventive concept is not limited to the image scanner occupying just the first and second example positions disclosed herein. The image scanner 540 may in fact occupy an unlimited number of positions on the track 550 without departing from the scope or spirit of the present general inventive concept. Accordingly, the first and second positions discussed herein are merely examples used for the sake of reference.

Still referring to FIG. 5, image scanner 540 and gantry 210 cooperate to simultaneously capture images of and treat the treatment volume 302 with particle therapy by performing a series of operations, including positioning operations. For example, after receiving a patient 301 having a treatment volume 302, such as a tumor, onto the treatment bed 236A in the treatment room 210A, the image scanner 540′ may be selectively positioned on the track 550 to occupy the first position proximate the patient's treatment volume 302 using the transport device 552, as illustrated by the dotted lines in FIG. 4. In embodiments where the image scanner 540′ includes a toroidal frame, such as in the example embodiment illustrated, the treatment bed 236A will be received through the central opening of the image scanner's frame. While occupying the first position, the image scanner 540′ may capture at least a first image of the treatment volume 302. In some embodiments, the first image is a CT image, which has been captured using the image scanner's x-ray tube and x-ray detector array.

After taking at least a first image, the image scanner 540 may be selectively repositioned by the transport device 552 to occupy a second position such that the image scanner 540 is not interposing the treatment nozzle 232A and the patient's treatment volume 302, as depicted by the solid lines in FIG. 4. In the illustrated example embodiment, the image scanner is not capturing images of the treatment volume 302 while occupying the second position. Instead, the beamline 215 may be directed to the treatment volume 302 without having to penetrate the image scanner 540.

In some example embodiments, the image scanner 540 may again be selectively positioned along the track 550 using the transport device 552, such that the image scanner 540′ once again occupies the first position. While occupying the first position, the image scanner 540′ may capture at least a second image of the treatment volume 302. In some embodiments, the second image is a PET image, which has been captured using the image scanner's PET detector arrays.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A simultaneous imaging and particle therapy treatment system comprising:
a means for generating a particle beamline;
a treatment bed to receive and support a patient having a treatment volume;
a gantry capable of receiving the particle beamline from the generating means and redirecting the beamline to the patient's treatment volume, the gantry capable of rotating about the treatment bed with a rotational axis substantially coplanar with the treatment bed and redirecting the beamline to encounter the treatment volume substantially perpendicular to the gantry's axis of rotation;
an image scanner having a plurality of detector arrays radially positioned around the treatment bed to capture images of the treatment volume; and
whereby the scanner and gantry are capable of simultaneously capturing images of and treating the treatment volume with particle therapy.

2. The system of claim 1, wherein the image scanner includes CT imaging components, PET imaging components, or both.

3. The system of claim 1, further comprising:
a means provided to the scanner for selectively positioning the scanner relative the treatment bed and/or the particle beamline.

4. The system of claim 3, wherein the positioning means comprises:
a track provided substantially parallel to the treatment bed, the scanner coupled to the track; and
a transport device coupled to the scanner to selectively position the scanner along the track.

5. The system of claim 3, wherein the positioning means comprises a non-linear actuator.

6. The system of claim 1, wherein the image scanner is capable of capturing images of the treatment volume while the beamline is encountering the treatment volume.

7. The system of claim 1, wherein the image scanner includes at least one treatment port through which the beamline passes to encounter the treatment volume.

8. The system of claim 1, further comprising an environmental chamber interposing the gantry and the patient through which the beamline passes before encountering the treatment volume.

9. The system of claim 8, further comprising a helium source in fluid communication with the environmental chamber.

10. The system of claim 8, further comprising a vacuum device in fluid communication with the environmental chamber.

11. The system of claim 8, wherein the environmental chamber is coupled to the gantry, the environmental chamber being selectively positionable relative the gantry between a first extended position and a second retracted position.

12. The system of claim 1, wherein a centerpoint of the plurality of radially positioned detector arrays is the treatment volume and at least one detector array is positionable adjacent the redirected beamline while the beamline is encountering the treatment volume.

13. The system of claim 1, wherein the gantry further defines an isocenter, the isocenter being an intersection of the redirected particle beamline and the gantry's axis of rotation, the scanner being positionable such that the isocenter is a centerpoint of the plurality of radially positioned detector arrays.

14. The system of claim 13, wherein the image scanner occupies a position such that the isocenter is the centerpoint of the plurality of radially positioned detector arrays while the particle beamline is encountering the treatment volume.

15. A simultaneous particle therapy treatment and imaging method comprising:
positioning on a treatment bed a patient containing a treatment volume, the treatment bed cooperating with a gantry, the gantry receiving a beamline from a particle generator means and redirecting the beamline to the treatment volume, the treatment bed further cooperating with an image scanner having a plurality of detector arrays radially positioned around the treatment bed to capture images of the treatment volume while the patient is on the treatment bed;
capturing a first image of the treatment volume using the image scanner;
directing a particle beamline to encounter the treatment volume;
capturing a second image of the treatment volume using the image scanner; and
whereby the capturing a first image operation, the directing operation, and the capturing a second image operation are performed simultaneously.

16. The method of claim 15, wherein the gantry rotates about the treatment bed with an axis of rotation substantially coplanar with the treatment bed, the gantry further redirecting the particle beamline to encounter the treatment volume substantially perpendicular to the axis of rotation, the gantry further defining an isocenter, the isocenter being the intersection of the redirected particle beamline and the axis of rotation, the method further comprising:
positioning the scanner to occupy a first position relative the treatment bed such that the isocenter is a centerpoint of the plurality of radially positioned detector arrays;
positioning the scanner to occupy a second position relative the treatment bed such that the isocenter is not the centerpoint of the plurality of radially positioned detector arrays; and
whereby the taking a first image operation and the taking a second image operation are performed by the scanner while occupying the first position, and the directing operation is performed by the gantry while the scanner occupies the second position.

17. The method of claim 15, wherein the image scanner captures CT images, PET images, or both.

18. The method of claim 17, wherein the first image is a CT image and the second image is a PET image.

19. The method of claim 15, wherein the capturing a second image operation is performed during the directing operation.
